(19) United States
(12) Patent Application Publication
(10) Pub. No.: US 2005/0181057 A1
(43) Pub. Date: Aug. 18, 2005

(54) Vaginal lubricant

(76) Inventors: Paul K. Rosenberg, Glen Ellyn, IL (US); Chim Potini, Bloomington, IL (US)

Correspondence Address: SONNENSCHEIN NATH & ROSENTHAL LLP, P.O. BOX 061080, WACKER DRIVE STATION, SEARS TOWER, CHICAGO, IL, 60606-1080, US

(21) Appl. No.: 10/778,905

(22) Filed: Feb. 13, 2004

(51) Int. Cl. A61K 9/14

(52) U.S. Cl. 424/488

(57) ABSTRACT

A vaginal lubricant comprises (i) water, (ii) a DHEA salt, (iii) a polyether, and (iv) a lubricant. The pH of the vaginal lubricant is at least 5.6.

### BACKGROUND

Vaginal dryness is a common problem which can cause physical and emotional distress in women (E. Key, Nurs. Stand, 5:24-27 (1991)). Inadequate lubrication due to vaginal dryness can also result in dyspareunia (characterized by difficult or painful sexual intercourse), a condition thought to affect approximately 40% of women; it has been estimated that over 40 million women will suffer dyspareunia at some time in their lives (S. Kelly, Clinical Practices and Sexuality 8(8):2 (1992)). Vaginal dryness is one of the most common symptoms of menopause. Vaginal dryness is often seen after childbirth or in women suffering from diseases, such as diabetes mellitus and autoimmune disorders. Treatments for various conditions, such as chemotherapy or radiotherapy for cancer, can cause vaginal dryness. Vaginal dryness can have a significant impact on the quality of life of women by adversely affecting their attitude toward intimate contact, a necessary part of healthy relationships.

Current methods for treating vaginal dryness include applying lubricating agents such as lubricating creams, jellies and topical estrogen creams; and hormone replacement therapy. Hormone replacement therapy is also effective for treating vaginal atrophy and dryness, but has several contraindications and unwanted side effects. Water-based lubricating composition are commercially available, for example, ASTROGLIDE® and K-Y JELLY®, as well as silicone-based lubricants. ASTROGLIDE® is typical of the water-based lubricants, which is a clear solution available as either a liquid or a gel. ASTROGLIDE® Liquid contains purified water, glycerin, propylene glycol, polyquaternium 15, methylparaben and propylparaben. ASTROGLIDE® Gel contains purified water, glycerin, hydroxyethyl cellulose, chlorhexidine gluconate, methylparaben, glucono delta lactone, and sodium hydroxide. These compositions have a slightly acidic pH of 5.5, to match normal body fluids, and to inhibit the growth of microorganisms, particularly yeast.

DHEA (dehydroepiandrosterone) is a steroid found in the body, and is believed to primarily function as a precursor for the synthesis of testosterone and estrogen. As an androgenic steroid, DHEA would be expected to improve libido in women. Prior to its release into the bloodstream, the vast majority of newly synthesized DHEA becomes sulfated (DHEA-S). The conjugated steroid DHEA-S is a secretory product of the adrenal gland in man and certain primates. DHEA-S represents the major steroid hormone in the circulation of humans, and is converted to DHEA via a sulfatase. Therapeutic uses for DHEA and its analogs have been reported for diabetes, dry skin, ocular hypertension, obesity, and retroviral infections. Illustrative of these reports are U.S. Pat. Nos. 4,395,408; 4,518,595; 4,542,129; 4,617,299; 4,628,052; and 4,666,898.

### SUMMARY

In a first aspect, the present invention is a vaginal lubricant, comprising (i) water, (ii) a DHEA salt, (iii) a polyether, and (iv) a lubricant. The pH of the vaginal lubricant is at least 5.6.

In a second aspect, the present invention is a vaginal lubricant, comprising (i) water, (ii) 5-25 mg/ml DHEA equivalents of a DHEA sulfate salt, (iii) 1.0-5.0 w/w % polyethylene glycol, (iv) glycerin and propylene glycol in a total amount of at most 5 w/w %, (v) triethanolamine, (vi) 0.01 to 0.1 w/w % hydroxyethyl cellulose, (vii) 0.2 to 1.5 w/w % methyl methacrylate, and (viii) methyl paraben and propyl paraben in a total amount of 0.01 to 5.0 w/w %.

In a third aspect, the present invention is a method of making a vaginal lubricant, comprising mixing together ingredients comprising: (i) water, (ii) a DHEA salt, (iii) a polyether, and (iv) a lubricant. The pH of the vaginal lubricant is at least 5.6.

In a fourth aspect, the present invention is a method of making a vaginal lubricant, comprising mixing together ingredients comprising: (i) water, (ii) 5-25 mg/ml DHEA equivalents of a DHEA sulfate salt, (iii) 1.0-5.0 w/w % polyethylene glycol, (iv) glycerin and propylene glycol in a total amount of at most 5 w/w %, (v) triethanolamine, (vi) 0.01 to 0.1 w/w % hydroxyethyl cellulose, (vii) 0.2 to 1.5 w/w % methyl methacrylate, and (viii) methyl paraben and propyl paraben in a total amount of 0.01 to 5.0 w/w %.

### DETAILED DESCRIPTION

It was desired to include DHEA in a water-based vaginal lubricant as it is hoped that it will restore vaginal moisture, and maintain and improve vaginal health and libido. However, DHEA is a hydrophobic compound and is therefore not significantly soluble in water, making it incompatible with water-based vaginal lubricants. The present invention makes use of the discovery that in order to include DHEA in a water-based vaginal lubricant, a DHEA salt must be used. Furthermore, even when a DHEA salt is used, a polyether must also be included to obtain complete dissolution of the DHEA salt, to produce a clear solution. In addition, it has also been discovered that when a DHEA salt is included in a water-based vaginal lubricant, the pH of the composition must be increased beyond the pH found in other vaginal lubricants, such as ASTROGLIDE®, to avoid creating a stinging sensation upon application.

The present invention includes a clear water-based vaginal lubricant containing a DHEA salt, which provides lubrication upon topical application, for relief of vaginal dryness. This composition contains a DHEA salt, at least one polyether, at least one lubricant, and optionally one or more pH adjusters, lubrication enhancers, thickeners, preservatives, fragrances, and dyes or colorants.

A DHEA salt has superior solubility in water as compared to DHEA. The DHEA salt is a pharmaceutically acceptable salt, such as alkali metal salts of DHEA sulfate, for example the sodium and/or potassium salt. A “pharmaceutically acceptable” compounds means that the desired activity of the composition is retained upon addition of the compound, and the compound does not impart undesired side-effects, such as toxicity, or an unpleasant odor. The DHEA salt is preferably present in an amount of 5-25 mg/ml DHEA equivalents, more preferably 7-20 mg/ml DHEA equivalents, most preferably 8-15 mg/ml DHEA equivalents.

In order to increase the solubility of the DHEA salt in the vaginal lubricant, to form a clear composition, the DHEA salt is dissolved in water and mixed with at least one polyether. The solution is then homogenized and filtered, before being mixed with other ingredients. Examples of polyethers include polyethylene glycol, such as PEG 400 and PEG 200. Other suitable polyethers include polypropylene glycol and polybutylene glycol. The amount of polyether present is sufficient to produce a clear composition, preferably a clear stock solution of the DHEA salt. Preferably, the concentration of the polyether in the stock solution containing water and a DHEA salt, is up to 50 w/w %, more preferably 1-30 w/w %, and most preferably 5-15 w/w %. In the vaginal lubricant, preferably the concentration of the polyether is 1-10 w/w %.

A lubricant is present to provide lubrication. Lubricants include hydrophilic polyols, such as glycerine and propylene glycol. A hydrophilic polyol is a polyol with a solubility in water at 25° C. of at least 0.1 g/L. One or more lubricants are included, preferably in a concentration of up to 5 w/w %, more preferably 0.14 w/w %, and most preferably 0.5-2.5 w/w %.

The pH of the vaginal lubricant must be great than 5.5, or a stinging sensation is produced upon topical application. Preferably, the pH is 5.6-6.5, more preferably 5.8-6.3, and most preferably 5.9-6.1. The pH of the present invention is distinct from the more acidic pH of ASTROGLIDE®, which has a pH of 5.5.

The pH of the vaginal lubricant is adjusted by addition of one or more pH adjusters, which are pharmaceutically acceptable acidic or basic compounds. Examples include common physiological buffers, acids such as hydrochloric acid, and bases such as sodium hydroxide and triethanolamine. The pH adjusters are used in an amount sufficient to adjust the pH to the desired value, preferably 1-5 w/w %.

Preferably, the vaginal lubricant includes one or more lubrication enhancers, which increase the lubrication provided by the composition, when at least one lubricant is already present. Lubrication enhancers are typically hydrophilic polymers, preferably derivatives of cellulose, such as hydroxyethyl cellulose. Preferably, the lubrication enhancers are present in an amount of 0.01 to 0.1 w/w %, more preferably 0.03 to 0.08 w/w %, and most preferably 0.4 to 0.6 w/w %.

Preferably, the vaginal lubricant includes one or more thickeners, which thicken the composition for ease of application, and to retain it in the general location of application. Thickeners include water-soluble polymers, such as acrylates, for example methyl methacrylate, acrylate copolymers and carbones. The amount included depends on the desired viscosity of the vaginal lubricant, with less used to produce a liquid composition, or more used to produce a gel-like composition. Preferably, the thickener is present in an amount of 0.2 to 1.5 w/w %, more preferably 0.35 to 1.3 w/w %, and most preferably 0.5 to 0.9 w/w %.

Preferably, the vaginal lubricant includes one or more preservatives. Examples of preservatives include methyl paraben and propyl paraben. Preferably, the preservative is present in an amount of 0.01 to 5.0 w/w %, more preferably 0.1 to 1.0 w/w %, and most preferably 0.3 to 0.8 w/w %.

Although not preferred, the vaginal lubricant may include one or more fragrances, flavorings, dye or colorants, of the types that are approved for use in cosmetic and pharmaceutical compositions.

### EXAMPLE

The following is an example of a composition for a vaginal lubricant:

| | | |
|---|---|---|
| DHEA, sodium sulfate salt (Carmel | 10 | mg/ml* |
| Olifins, Jerusalem, Israel) | | |
| PEG 400 (polyethylene glycol) | 5.0 | w/w % |
| Glycerin, USP | 1.0 | w/w % |
| Propylene glycol, USP | 1.0 | w/w % |
| Triethanol amine | 0.6 | w/w % |
| Hydroxyethyl Cellulose, USP | 0.05 | w/w % |
| Carbopol 980 (methyl methacrylate; | 0.7 | w/w % |
| B. F. Goodrich) | | |
| Methyl Paraben | 0.5 | w/w % |
| Propyl Paraben | 0.5 | w/w % |

| | |
|---|---|
| Water | remainder |

*DHEA equivalents.
**Present in an amount sufficient to produce a clear composition.

1. A vaginal lubricant, comprising:
(i) water,
(ii) a DHEA salt,
(iii) a polyether, and
(iv) a lubricant,
wherein the pH of the vaginal lubricant is at least 5.6.

2. The vaginal lubricant of claim 1, wherein the DHEA salt comprises DHEA sodium sulfate salt.

3. The vaginal lubricant of claim 1, wherein the polyether comprises polyethylene glycol.

4. The vaginal lubricant of claim 1, wherein the lubricant comprises glycerin.

5. The vaginal lubricant of claim 1, wherein the lubricant comprises propylene glycol.

6. The vaginal lubricant of claim 1, further comprising a pH adjuster.

7. The vaginal lubricant of claim 6, wherein the pH adjuster comprises triethanolamine.

8. The vaginal lubricant of claim 1, further comprising a lubrication enhancer.

9. The vaginal lubricant of claim 8, wherein the lubrication enhancer comprises hydroxyethyl cellulose.

10. The vaginal lubricant of claim 1, further comprising a thickener.

11. The vaginal lubricant of claim 10, wherein the thickener comprises methyl methacrylate.

12. The vaginal lubricant of claim 1, further comprising a preservative.

13. The vaginal lubricant of claim 12, wherein the preservative comprises methyl paraben.

14. The vaginal lubricant of claim 12, wherein the preservative comprises propyl paraben.

15. A vaginal lubricant, comprising:
(i) water,
(ii) 5-25 mg/ml DHEA equivalents of a DHEA sulfate salt,
(iii) 1.0-5.0 w/w % polyethylene glycol,
(iv) glycerin and propylene glycol, in a total amount of at most 5 w/w %,
(v) triethanolamine,
(vi) 0.01 to 0.1 w/w % hydroxyethyl cellulose,
(vii) 0.2 to 1.5 w/w % methyl methacrylate, and
(viii) methyl paraben and propyl paraben, in a total amount of 0.01 to 5.0 w/w %.

16. The vaginal lubricant of claim 15, wherein the DHEA sulfate salt comprises DHEA sodium sulfate salt.

17. A method of making a vaginal lubricant, comprising mixing together ingredients comprising:
(i) water,
(ii) a DHEA salt,
(iii) a polyether, and
(iv) a lubricant,
wherein the pH of the vaginal lubricant is at least 5.6.

18. The method of claim 17, wherein the ingredients further comprise:
(v) a pH adjuster,
(vi) a lubrication enhancer,
(vii) a thickener, and
(viii) a preservative.

19. A method of making a vaginal lubricant, comprising mixing together ingredients comprising:
(i) water,
(ii) 5-25 mg/ml DHEA equivalents of a DHEA sulfate salt,
(iii) 1.0-5.0 w/w % polyethylene glycol,
(iv) glycerin and propylene glycol, in a total amount of at most 5 w/w %,
(v) triethanolamine,
(vi) 0.01 to 0.1 w/w % hydroxyethyl cellulose,
(vii) 0.2 to 1.5 w/w % methyl methacrylate, and
(viii) methyl paraben and propyl paraben, in a total amount of 0.01 to 5.0 w/w %.

20. A vaginal lubricant prepared by the method of claim 17.

21. A vaginal lubricant prepared by the method of claim 19.
